(19) United States
(12) Patent Application Publication
(10) Pub. No.: US 2005/0197303 A1
(43) Pub. Date: Sep. 8, 2005

(54) Combination of loteprednol etabonate and tobramycin for topical ophthalmic use

(75) Inventor: Ramesh Krishnamoorthy, Cary, NC (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/049,355

(22) Filed: Feb. 1, 2005

Related U.S. Application Data
(63) Continuation-in-part of application No. 10/698,322, filed on Oct. 31, 2003.

(51) Int. Cl. A61K 31/704; A61K 31/573; A61K 31/14

(52) U.S. Cl. 514/35; 514/39; 514/41; 514/171; 514/643

(57) ABSTRACT

This invention relates to formulations for topical use comprising antibiotics in combination with anti-inflammatory steroids for treating ophthalmic infections and attendant inflammation. More specifically, this invention relates to pharmaceutical ophthalmic formulations comprising a pH stabilizing amount of an aminoglycoside and a steroid in a pharmaceutically acceptable vehicle.

### PRIORITY CLAIM

This application is a continuation in part of U.S. application Ser. No. 10/698,322 filed Oct. 31, 2003, the entire contents of which are incorporated by reference herein.

### FIELD OF THE INVENTION

This invention relates to formulations for topical use comprising antibiotics in combination with anti-inflammatory steroids for treating ophthalmic infections and attendant inflammation. More specifically, this invention relates to pharmaceutical ophthalmic formulations comprising a pH stabilizing amount of an aminoglycoside and a steroid.

### BACKGROUND OF THE INVENTION

Topical steroids such as corticosteroids are commonly used for anti-inflammatory therapy of the eye, especially for treating inflammatory conditions of the palpebral or bulbar conjunctiva, cornea and anterior segment of the globe. Common therapeutic applications for steroids include allergic-conjunctivitis, acne rosacea, superficial punctate keratitis and iritis cyclitis. Steroids also are used to ameliorate inflammation associated with corneal injury due to chemical or thermal burns, or penetration of foreign bodies. Such conditions may result from surgery, injury, allergy or infection to the eye and can cause severe discomfort.

Despite their therapeutic advantages, topical ocular use of corticosteroids is associated with a number of complications, including posterior subcapsular cataract formation, elevation of intraocular pressure, secondary ocular infection, retardation of corneal wound healing, uveitis, mydriasis, transient ocular discomfort and ptosis. Numerous systemic complications also may arise from the topical ocular application of corticosteroids. These complications include adrenal insufficiency, Cushing's syndrome, peptic ulceration, osteoporosis, hypertension, muscle weakness or atrophy, inhibition of growth, diabetes, activation of infection, mood changes and delayed wound healing.

Topical steroids for treating ocular inflammations can be based on soft drugs. Soft drugs, as is known in the art, are designed to provide maximal therapeutic effect and minimal side effects. By one approach, synthesis of a “soft drug” can be achieved by structurally modifying a known inactive metabolite of a known active drug to produce an active metabolite that undergoes a predictable one-step transformation in-vivo back to the parent, inactive metabolite (see, U.S. Pat. Nos. 6,610,675, 4,996,335 and 4,710,495 for soft steroids). “Soft drugs” therefore are biologically active chemical components characterized by predictable in vivo metabolism to non-toxic derivatives after they provide their therapeutic effect. Formulations of cortico steroids suitable for ophthalmic use are known. For example, U.S. Pat. Nos. 4,710,495, 4,996,335, 5,540,930, 5,747,061, 5,916,550, 6,368,616 and 6,610,675, the contents of each of which is incorporated by reference herein, describe soft steroids and formulations containing soft steroids.

Antibiotic agents for use in treating ophthalmic infections are also known. For example penicillins, cephalosporins and aminoglycosides such as amikacin, gentamicin and tobramycin are known to be useful in treating infections of the eye. Tobramycin is commercially marketed and well recognized as an effective antibiotic. This particular anti-infective is recognized as active against the common bacterial eye pathogens: Staphylococci, including S. aureus and S. epidermidis, including penicillin resistant strains, Streptococci, including S. pneumoniae, Pseudomonas aeruginosa, Escherichia coli, Klebsiella pneumoniae, Enterobacter aerogenes, Proteus mirabilis, Morganella morganii, Haemophilus influenzae, H. aegyptius, Acinetobacter calcoaceticus and some Neissaria species. Tobramycin's antimicrobial activity is provided by its ability to bind with the 30S ribosomal subunit and alter protein synthesis, thus leading to the death of the microbial organism.

It has previously been suggested that the steroid loteprednol etabonate (LE) can be combined with antibiotics such as tobramycin. However, there has been no suggestion of the amount of tobramycin to be used in combination with LE to provide a desired therapeutic effect of both active agents. There has also been no detailed description of a combined formulation having satisfactory properties for storage and use of the combination of tobramycin and LE.

It is known that formulations containing steroids can experience stability problems. Such stability problems include clumping and other undesirable changes upon storage. U.S. Pat. No. 5,916,550 describes the use of C₂-C₇ aliphatic amino acids to control pH depression of aqueous suspensions of LE on prolonged storage. Therefore, the need to provide pharmaceutical formulations of steroids that are stable upon storage is well recognized. One of the factors used to evaluate stability of pharmaceutical formulations is pH. When there is a dramatic change in the pH of a pharmaceutical formulation over time, the ability of the formulation to be effectively stored and retain its pharmaceutical activity after storage becomes questionable. It is known to add buffers to certain pharmaceutical formulations in an effort to maintain the stability of the formulation during storage. Examples of buffers include borate buffers, phosphate buffers, etc. Although these buffers are useful in stabilizing pH, they do not demonstrate pharmaceutical activity. Therefore, it would be desirable to use a single material to provide pH stabilizing activity and desired anti-infection activity to pharmaceutical ophthalmic formulations containing a steroid.

### SUMMARY OF THE INVENTION

It has surprisingly been discovered that the an aminoglycoside, such as tobramycin, when present in a pH stabilizing amount, helps to stabilize steroid containing formulations over time to provide better storage characteristics.

This invention provides novel compositions of matter containing a combination of water-soluble and water-insoluble drugs suitable for therapeutic use. The invention provides pH stable aqueous suspensions of water-insoluble drugs that remain in such a state even after extended periods of storage.

More particularly, the invention is directed to aqueous suspensions of steroids such as loteprednol etabonate in combination with aminoglycosides such as tobramycin suitable for therapeutic use in the eye, ear, or nose. The aqueous suspensions of steroid and aminoglycoside are surprisingly pH stable and can remain in a pH stable state for extended periods of storage.

Formulations comprising the broad spectrum aminoglycoside antibiotic in combination with a steroid loteprednol provide pharmaceutical ophthalmic formulations that not only allow for the simultaneous treatment of inflammation and infection in a patient in need of treatment thereof, but also results in a pharmaceutical ophthalmic formulation having increased stability, as measured by decreased change in pH as compared to similar steroid formulations that do not contain a pH stabilizing amount of an aminoglycoside.

Further provided herein is a topical eye drop medication indicated for steroid-responsive inflammatory ocular conditions for which a corticosteroid is indicated and where superficial bacterial ocular infection or risk of bacterial ocular infection exists. The medication comprises a steroid/aminoglycoside ophthalmic suspension. The use of this medication is indicated where the risk of superficial ocular infection is high or where there is an expectation that potentially dangerous numbers of bacteria will be present in the eye.

Also provided herein is a therapeutically effective composition comprising a steroid in an amount effective to provide a therapeutic benefit to a patient to whom the composition is administered and a pH stabilizing amount of an aminoglycoside, wherein the aminoglycoside is present in an amount effective to stabilize the pH of the composition relative to the pH of a similar formulation without the aminoglycoside.

Also provided herein is a method of treating a patient having inflammatory ocular conditions for which a corticosteroid is indicated and where superficial bacterial ocular infection or risk of bacterial ocular infection exists, the method comprising topically applying to a patient in need of treatment thereof therapeutic amount of a pharmaceutical composition comprising a ph stabilizing amount of a broad spectrum aminoglycoside antibiotic in combination with the a steroid.

Having briefly summarized the invention, the invention will now be described in detail by reference to the following specification and non-limiting examples. Unless otherwise specified, all percentages are by weight and all temperatures are in degrees Celsius.

### DETAILED DESCRIPTION OF THE INVENTION

Therapeutic suspensions of steroids for ophthalmic or otolaryngological uses are made by aseptic preparation. Purity levels of all materials employed in the suspensions of the invention exceed 98%. The suspensions of the invention are prepared by thoroughly mixing the steroid (component (A)), aminoglycoside (component (B)), suspending agent (component (C)), and surface active agent (component (D)). Optionally, tonicity agents (component (E)) and preservatives (component (F)) may be included.

Steroids of component (A), preferably soft steroids, most preferably LE, can be employed. Also other steroids such as beclomethasone, betamethasone, fluocinolone, fluorometholone, exednisolone, prednisolone and rimexolone may be employed. The suspensions of component (A) of the invention have a particle size of about 0.1-30μ, preferably about 1-20μ, most preferably about 2-10 microns in mean diameter. LE in this size range is commercially available from suppliers such as the Sipsy Co., (Avrille, France).

The aminoglycoside component (B) is pharmaceutical grade. Aminoglycosides are a well-characterized family of antimicrobial agents and include, for example, gentamicin, neomycin, paromomycin, kanamycin, tobramycin, netilmicin and amikacin. Tobramycin of this grade is commercially available from suppliers such as the Biogal Pharmaceutical Works (Debrecen, Hungary). Component (B) is preferably present in an amount that is effective to stabilize the pH of the composition relative to the pH of a similar composition without Component (B). Therefore the amount of Component (B) may vary depending upon the individual composition. Determining a pH stabilizing amount of an aminoglycoside for a particular composition can be readily achieved through routine experimentation and is within the purview of one skilled in the art.

The nonionic polymer of component (C) can be any nonionic water-soluble polymer. Typical compounds such as PVP, PVA, HPMC or dextran can be used at a concentration of about 0.01-2%, and preferably between about 0.4 to 1.5%, and more preferably between 0.4 to 1%. Viscosity increased above that of simple aqueous solutions may be desirable to increase ocular absorption of the active compound, to decrease variability in dispensing the formulation, to decrease physical separation of components of a suspension or emulsion of the formulation and/or to otherwise improve the ophthalmic formulation. Such viscosity builder agents include as examples polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art. Povidone is preferably used as a suspending agent in the finished product and the water-soluble grades are routinely used in pharmaceuticals as a viscosity enhancing agent. The viscosity of aqueous solutions of the water-soluble grades of povidone depends on the average molecular weight. A subtle change in the grade and concentration of the suspending agent can yield the desired characteristics. Povidone comes in a variety of grades, of which some are water soluble. Povidone K-90 is the highest molecular weight water-soluble viscosity grade Povidone. This material is listed as Povidone, USP 90,000. The high molecular grade povidone dissolves much more slowly than the lower molecular weight grade.

Component (D) is a surface-active agent that is acceptable for ophthalmic or otolaryngological uses. Preferably, this surfactant is non-ionic. Useful surface active agents include but are not limited to polysorbate 80, tyloxapol, TWEEN 80 (ICI America Inc., Wilmington, Del.), PLURONIC F-68 (from BASF, Ludwigshafen, Germany) and the poloxamer surfactants. These surfactants are nonionic alkaline oxide condensates of an organic compound that contains hydroxyl groups. The concentration in which the surface active agent may be used is only limited by neutralization of the bactericidal effects on the accompanying preservatives, or by concentrations that may cause irritation. Preferably, the concentration of component (D) is about 0.05 to 1%, and more preferably 0.1 to 0.6% by weight based on the weight of the suspension. Compositions of the present invention having a molar ratio of (A):(C):(D) between about 1:20:1 and about 1:0.01:0.5 are entirely suitable.

The tonicity agents of component (E) can be nonionic diols, preferably glycerol, in sufficient amounts to achieve isotonicity. The nonionic tonicity agents can be present in an amount of about 2 to 2.8% by weight, and preferably about 2.2 to 2.6%.

The nonionic polymeric compounds of component (C), and the surface active agents of component (D) have good solubility in water, have sufficient number of hydroxyl groups to interact with the steroid, and have mild effects on the viscosity of the suspension. Final viscosity should not exceed 80-centipoise.

The suspensions of the invention also may include additional therapeutic drugs such as drugs for treating glaucoma, anti-inflammatory drugs, anti-cancer drugs, anti-fungal drugs and anti-viral drugs. Examples of anti-glaucoma drugs include but are not limited to timolol-base, betaxalol, athenolol, levobanolol, epinenephrin, dipivalyl, oxonolol, acetazilumide-base and methazalomide. Examples of anti-inflammatory drugs include but are not limited to non-steroids such as piroxicam, indomethacin, naproxen, phenylbutazone, ibuprofen and diclofenac.

Health regulations in various countries generally require that ophthalmic preparations shall include a preservative. Many well known preservatives that have been used in ophthalmic preparations of the prior art, however, cannot be used in the preparations of the invention, since those preservatives may no longer be considered safe for ocular use, or may interact with the surfactant employed in the suspension to form a complex that reduces the bactericidal activity of the preservative.

The preservatives of component (F) employed in the suspensions of the invention therefore are chosen to not interact with the surface active agent to an extent that the preservatives are prevented from protecting the suspension from microbiological contamination. In a preferred embodiment benzalkonium chloride may be employed as a safe preservative, most preferably benzalkonium chloride with EDTA. Other possible preservatives include but are not limited to benzyl alcohol, methyl parabens, propyl parabens, thimerosal, chlorbutanol and benzethonium chlorides. Typically such preservatives are employed at a level of from 0.001% to 1.0% by weight. Preferably, a preservative (or combination of preservatives) that will impart standard antimicrobial activity to the suspension and protect against oxidation of components (A)-(E) is employed.

In forming compositions for topical administration, the mixtures are preferably formulated as 0.01 to 2.0 percent by weight solutions in water at a pH of 4.5 to 8.0 (figures relate to combined presence of loteprednol etabonate and tobramycin). While the precise regimen is left to the discretion of the clinician, it is recommended that the resulting solution be topically applied by placing one drop in each eye two times a day.

A bioavailability study of LE-tobramycin vs. LOTEMAX loteprednol etabonate composition demonstrated that in the intend to treat population bioequivalence was met at both the 40 and 60 minute sampling periods. Thus, the inclusion of tobramycin does not alter the ocular bioavailability of loteprednol etabonate. A microbial kill rate study was undertaken to demonstrate antimicrobial equivalence between loteprednol etabonate and tobramycin ophthalmic suspension, 0.5%/0.3% and tobramycin ophthalmic solution, USP 0.3%. The methods employed were based on USP anti-microbial effectiveness procedures for preparation of inoculum and challenge concentration of test organisms. The anti-microbial activity of both products was demonstrated against 22 organisms. The in vitro study demonstrated that tobramycin has equivalent anti-microbial activity as a single agent and when in combination with loteprednol etabonate.

Without further elaboration, it is believed that one skilled in the art can, using the preceding description, utilize the present invention to its fullest extent. The following preferred specific embodiments therefore are to be construed as merely illustrative, and not limitative of the remainder of the disclosure in any way whatsoever. In the following examples, all temperatures are set forth in degrees Celsius; unless otherwise indicated, all parts and percentages are by weight.

### EXAMPLES

A study was undertaken to compare a standard LE-tobramycin composition having different concentrations of Povidone and different types of Povidone. The example compositions also contained standard pharmaceutical components. Examples III and VI were used as controls (no tobramycin) to observe the effect of tobramycin on the pH of the composition. The materials were mixed with purified water and held at a temperature of 28° C. to represent room temperature stability and 40° C. to represent accelerated stability. The results are given in the tables below.

STABILITY OF LE-TOBRAMYCIN MATRIX (with different viscosity)
28° C.

| | Time | Tobra. | LE | |
|---|---|---|---|---|
| | (month) | (mg/ml) | (mg/ml) | pH |
| I | 0 | 3.18 | 4.976 | 6.49 |
| 0.6% | 1 | 3.076 | — | 6.32 |
| PVP-C30 | 2 | 3.062 | 4.947 | 6.28 |
| | 3 | 3.113 | 5.484 | 6.21 |
| | 6 | 3.003 | 5.155 | 6.17 |
| II | 0 | 3.165 | 5.241 | 6.47 |
| 1.5% | 1 | 3.04 | — | 6.3 |
| PVP-C30 | 2 | 2.995 | 5.08 | 6.19 |
| | 3 | 3.04 | 5.32 | 6.14 |
| | 6 | 3.008 | 5.514 | 6.087 |
| III | 0 | — | 5.576 | 5.98 |
| 1.5% | 1 | — | — | 5.54 |
| PVP-C30 | 2 | — | 5.411 | 5.06 |
| Control | 3 | — | 5.706 | 5.02 |
| | 6 | — | 5.134 | 4.8 |
| IV | 0 | 3.206 | 5.296 | 6.71 |
| 1.5% | 1 | 3.082 | 5.156 | 6.57 |
| PVP-K90 | 2 | 3.122 | 5.29 | 6.49 |
| | 3 | 3.21 | 5.306 | 6.47 |
| | 6 | 3.146 | 5.358 | 6.39 |
| V | 0 | 2.802 | 4.32 | 6.61 |
| 0.5% | 1 | 2.754 | 4.24 | 6.48 |
| PVP-K90 | 2 | 2.596 | 4.28 | 6.38 |
| | 3 | 2.811 | 4.336 | 6.34 |
| | 6 | 2.806 | 4.365 | 6.32 |
| VI | 0 | — | 5.426 | 6.61 |
| 1.5% | 1 | — | 5.638 | 5.033 |
| PVP-K90 | 2 | — | 5.67 | 4.71 |
| Control | 3 | — | 5.67 | 4.72 |
| | 6 | — | 5.727 | 4.43 |

STABILITY OF LE-TOBRAMYCIN MATRIX (with different viscosity)
40° C.

| | Time | Tobra. | LE | |
|---|---|---|---|---|
| | (month) | (mg/ml) | (mg/ml) | pH |
| I | 0 | 3.18 | 4.976 | 6.49 |
| 0.6% | 1 | 3.15 | — | 6.23 |
| PVP-C30 | 2 | 2.957 | 5.008 | 6.06 |
| | 3 | 3.036 | 5.067 | 5.95 |
| | 6 | | | |
| II | 0 | 3.165 | 5.241 | 6.47 |
| 1.5% | 1 | 3.019 | — | 6.21 |
| PVP-C30 | 2 | 2.91 | 5.16 | 5.89 |
| | 3 | 2.95 | 5.37 | 5.89 |
| | 6 | | | |
| III | 0 | — | 5.576 | 5.98 |
| 1.5% | 1 | — | — | 4.73 |
| PVP-C30 | 2 | — | 5.411 | 4.33 |
| Control | 3 | — | 5.473 | 4.11 |
| | 6 | — | | |
| IV | 0 | 3.206 | 5.296 | 6.71 |
| 1.5% | 1 | 2.94 | 5.336 | 6.47 |
| PVP-K90 | 2 | 2.84 | 5.209 | 6.13 |
| | 3 | 3.17 | 5.17 | 6.25 |
| | 6 | 3.212 | 5.178 | 6.097 |
| V | 0 | 2.802 | 4.32 | 6.61 |
| 0.5% | 1 | 2.588 | 4.284 | 6.38 |
| PVP-K90 | 2 | 2.64 | 4.21 | 6.21 |
| | 3 | 2.82 | 4.2 | 6.14 |
| | 6 | 2.767 | 4.267 | 6.04 |
| VI | 0 | — | 5.426 | 6.61 |
| 1.5% | 1 | — | 5.614 | 4.62 |
| PVP-K90 | 2 | — | 5.91 | 3.93 |
| Control | 3 | — | 5.85 | 3.83 |
| | 6 | — | 5.572 | 3.53 |

The above data represents the results of pH stability testing of various compositions having differing viscosity. PVP-C30 is Povidone having a molecular weight of around 30,000 and PVP-K90 is Povidone having a molecular weight of around 90,000. Both were obtained from the GAF Corporation, USA. In general a pH between 4.5 and 7.0 is considered acceptable for pharmaceutical ophthalmologic use of these compositions. The data demonstrates that compositions of the present invention having tobramycin display a more gradual decrease in pH over time and less of a total change in pH over time as compared to similar compositions which do not contain tobramycin.

The following example is a representative pharmaceutical composition of the invention for topical use where indicated against inflammation and infection.

### EXAMPLE 1

Ingredients (per mL)

Loteprednol etabonate 0.5% (5 mg)
Glycerin 2.5%
Povidone, K-90 0.6%
Tobramycin 0.3% (3 mg)
Benzalkonium Chloride 0.01%
Tyloxapol 0.05%
Edentate disodium 0.01%
Purified water (QS to 100%)
Sulfuric acid or sodium hydroxide (to adjust pH)

1. A composition for ophthalmic or otolaryngological use comprising:
a steroid having a particle size of 0.1 to 30 microns in diameter and a pH stabilizing amount of an aminoglycoside in a pharmaceutically acceptable vehicle.

2. The composition of claim 1 wherein when the steroid is dexamethasone the aminoglycoside is not tobramycin.

3. The composition of claim 1 wherein the steroid is selected from the group consisting of beclomethasone, betamethasone, fluocinolone, fluorometholone, exednisolone, loteprednol etabonate, prednisolone and rimexolone.

4. The composition of claim 1 wherein the aminoglycoside is selected from the group consisting of gentamycin, neomycin, paromomycin, kanamycin, tobramycin, netilmicin and amikacin.

5. The composition of claim 4 wherein the aminoglycoside is present in an effective anti-infection amount.

6. The composition of claim 1 further including a preservative for preventing microbial formation in said composition.

7. The composition of claim 6 wherein said preservative is benzalkonium chloride.

8. The composition of claim 7 further comprising disodium edentate.

9. The composition of claim 1 further comprising a nonionic polymer.

10. The composition of claim 9 wherein the nonionic polymer is selected from the group consisting of polyvinylpyrrolidone, polyvinyl alcohol, dextran and mixtures thereof.

11. The composition of claim 1 further comprising a surface active agent.

12. The composition of claim 11 wherein the surface active agent is tyloxapol and is present in an amount of about 0.1 to 0.6% by weight.

13. The composition of claim 1 further comprising an additional therapeutic drug in admixture with the steroid and aminoglycoside, wherein said additional therapeutic drug is selected from the group consisting of betaxalol, athenolol, levobanolol, epinenephrin, dipivalyl, oxonolol, acetazilumide-base, methazalomide, piroxicam, indomethacin, naproxen, phenylbutazone, ibuprofen, and diclofenac-acid.

13. A composition for ophthalmic or otolaryngological anti-inflammatory and anti-infection use comprising a nonionic polymer in an aqueous medium, a nonionic tonicity agent in an amount effective to achieve isotonicity, and a nonionic surface active agent in an amount sufficient to retain the polymer and tonicity agent in the aqueous medium, tobramycin; and a steroid having a particle size of 0.1 to 30 microns in diameter in an amount of about 0.2 to 2% by weight, wherein the composition further comprises a pH stabilizing amount of an aminoglycoside.

14. The composition of claim 13 wherein said nonionic tonicity agent is a nonionic diol and is present in an amount of about 2 to 2.8% by weight.

15. The composition of claim 13 wherein the nonionic polymer is present in an amount of about 0.2 to 2% by weight; the nonionic tonicity agent is present in an amount of about 2 to 2.8% by weight; and the nonionic surface active agent is present in an amount of about 0.05 to 1% by weight.

16. The composition of claim 13 further comprising a preservative of benzalkonium chloride, disodium edentate, and mixtures thereof in an amount of about 0.01 to 0.025% by weight.

17. The composition of claim 13 wherein the nonionic polymer is polyvinyl pyrrolidone and is present in an amount of about 0.4 to 1% by weight, the nonionic tonicity agent is mannitol or a diol and is present in an amount of about 2 to 2.8% by weight, and the nonionic surface active agent is tyloxapol and is present in an amount of about 0.1 to 0.6% by weight.

18. The composition of claim 1 wherein the steroid is present in an amount of between about 0.01 and 10% by weight.

19. The composition of claim 1 wherein the steroid is loteprednol etabonate and is present in an amount of between about 0.05 and about 10.0% by weight.

20. The composition of claim 13 wherein said nonionic polymer is a water soluble polymer selected from the group consisting of polyvinylpyrrolidone, polyvinyl alcohol, dextran and cyclodextrin.

21. The composition of claim 13 wherein the nonionic polymer is present in an amount of about 0.2 to 2% by weight.

22. The composition of claim 20, wherein said nonionic polymer is polyvinylpyrrolidone and is present in an amount of between about 0.3 to about 1.75% by weight.

23. The composition of claim 13 wherein said nonionic surface active agent is tyloxapol and is present in an amount of about 0.05 to about 1% by weight.

24. The composition of claim 13, wherein said nonionic surface active agent is a polyoxyethylene sorbitan mono-oleate ester.

25. The composition of claim 22 wherein the nonionic tonicity agent is present in an amount of between about 1 to about 7% by weight.

26. The composition of claim 25 wherein the nonionic tonicity agent is a nonionic polyol and is present in an amount of between about 1.5 to about 4% by weight.

27. The composition of claim 26 wherein the nonionic tonicity agent is glycerol or mannitol.

28. The composition of claim 13 further including a preservative for preventing microbial formation in said composition and is present in an amount of between about 0.0001 to about 0.025% by weight.

29. The composition of claim 28 wherein said preservative is selected from the group consisting of benzalkonium chloride, disodium edetate and mixtures thereof.

30. The composition of claim 13 further comprising an additional therapeutic drug in admixture with said soft steroid and tobramycin, wherein said additional therapeutic drug is selected from the group consisting of betaxolol, atenolol, levobunolol, epinephrin, dipivalyl, oxonolol, acetazolamide-base, methazolamide, piroxicam, indomethacin, naproxen, phenylbutazone, ibuprofen, and diclofenac.

31. The composition of claim 13 wherein the nonionic polymer is present in an amount of between about 0.2 to about 2% by weight; the nonionic tonicity agent is present in an amount of between about 1 to about 7% by weight; and the nonionic surface active agent is present in an amount of between about 0.05 to about 1% by weight.

32. The composition of claim 31 further comprising a preservative for preventing microbial formation in said composition and is present in an amount of about 0.0001 to 0.025% by weight.

33. A method for treating ophthalmic or otolaryngological inflammation and infection which comprises applying to inflamed tissue a the composition of claim 1; wherein said composition is applied in an amount effective to treat said inflammation and infection.
